(19) United States
(12) Patent Application Publication
(10) Pub. No.: US 2023/0138974 A1
(43) Pub. Date: May 4, 2023

(54) Polyherbal Self Micro-Emulsifying Delivery System (SMEDS) composition for multi-disease treatment

(71) Applicant: Barany Jeganatth, Reno, NV (US)

(72) Inventor: Barany Jeganatth, Reno, NV (US)

(21) Appl. No.: 17/972,633

(22) Filed: Oct. 25, 2022

Related U.S. Application Data
(60) Provisional application No. 63/275,364, filed on Nov. 3, 2021.

(51) Int. Cl. A61K 31/164; A61K 36/06; A61K 36/74; A61K 31/366; A61K 31/352; A61K 36/67; A61K 36/752; A61K 36/54; A61K 31/122; A61K 31/593

(52) U.S. Cl. CPC A61K 31/164; A61K 36/06; A61K 36/74; A61K 31/366; A61K 31/352; A61K 36/67; A61K 36/752; A61K 36/54; A61K 31/122; A61K 31/593

(57) ABSTRACT

The present invention relates to a polyherbal Pan PPAR and ECS Agonist composition with Antioxidant and Anti-inflammatory function to treat and prevent Chronic Pain, Alzheimer's Dementia, Hyperlipidemia, Depression, Anxiety, Inflammatory Disease and Metabolic Disorders. The composition comprises botanical mixture of Palmitoylethanolamide, Botanical Extracts (Nutmeg, Black Pepper, Holy Basil, Cinnamon, Cassia, Ginger, Rosemary, Terpene isolates of Beta Caryophyllene and Limonene), Carotene Complex, Coenzyme Q10, Tocotrienols, Vitamin D3, Vitamin K2-MK7, Piperine, Phyto Cannabinoids derived from Cannabis, Docosahexanoic acid (DHA) and or Alpha Linolenic Acid (ALA).

### TECHNICAL FIELD OF THE INVENTION

The present invention relates to a polyherbal Pan PPAR and ECS Agonist composition with Antioxidant and Anti-inflammatory function to treat and prevent Chronic Pain, Alzheimer's Dementia, Hyperlipidemia, Depression, Anxiety, Inflammatory Disease and Metabolic Disorders. The composition comprises botanical mixture of Palmitoylethanolamide (PEA), Botanical Extracts (Nutmeg, Black Pepper, Holy Basil, Cinnamon, Cassia, Ginger, Rosemary, Terpene isolates of Beta Caryophyllene and Limonene), Carotene Complex, Coenzyme Q10, Tocotrienols, Vitamin D3, Vitamin K2-MK7, Piperine, Phyto Cannabinoids derived from Cannabis, Docosahexanoic acid (DHA) and or Alpha Linolenic Acid (ALA).

### BACKGROUND OF THE INVENTION

PPAR agonists act upon the peroxisome proliferator-activated receptor (PPARs) and are used for the treatment of symptoms of the metabolic syndrome, such as increasing insulin sensitivity, lowering triglycerides and blood sugar. The main types of PPAR agonists are PPAR-alpha agonists, PPAR-gamma agonists and PPAR-delta agonists. PPARα (alpha) is the main target of fibrate drugs, they are used to treat cholesterol disorders and high triglycerides. PPARγ (gamma) is the main target of thiazolidinediones (TZDs), used for treatment of diabetes mellitus, insulin resistance and in treating hyperlipidaemia in atherosclerosis. PPARδ (Delta) is the main target of a synthetic compound named GW501516, which is currently under clinical investigations.

Synthetic compounds such as aleglitazar, muraglitazar and tesaglitazar are Dual PPAR agonists (PPAR Alpha and Gamma) that have failed clinical investigations due to higher incidence of all-cause mortality, cardio toxicity and myocardial infraction. PPAR Alpha and Gamma agonists are also being investigated for treatment of depression and drug addiction. Dual and Pan PPAR agonists (to activate all three PPAR Isoforms simultaneously) is currently under active investigation for treatment of a larger subset of the symptoms of the metabolic syndrome. There is an urgent need to develop PPAR ligands with fewer adverse effects.

The Endocannabinoid System (ECS) is actively involved in inflammation. Activation of all PPAR isoforms, primarily PPARα and γ, mediates some of the analgesic, neuroprotective, and anti-inflammatory effects of cannabinoids in the ECS along with activation of the cannabinoid CB₁ and CB₂ receptors and TRPV1 ion channels. Medications that impact the ECS, including PEA and the cannabinoids CBD and THC, offer significant benefit for pain driven by neuroinflammation and oxidative stress.

Cannabinoids refer to naturally occurring compounds found in cannabis and non-cannabis sources. The most common Cannabinoids used for medicinal values are tetrahydrocannabinol (THC), cannabigerol (CBG), Cannabigerolic acid (CBGA) and cannabidiol (CBD). An example of non-Cannabis derived Cannabinoids is Palmitoylethanolamide (PEA), which is synthesized from Palmitic Acid.

Tetrahydrocannabinol (THC) is the chemical responsible for most of marijuana's psychological effects. It acts much like the cannabinoid chemicals made naturally by the body, according to the National Institute on Drug Abuse (NIDA). It is chemically 6, 6, 9-trimethyl-3-pentyl-6a, 7, 8, 10a-tetrahydrobenzo[c]chromen-1-ol

Cannabigerol (CBG), is one of more than 120 identified cannabinoid compounds found in the plant genus Cannabis. Cannabigerol is the decarboxylated form of cannabigerolic acid, the parent molecule from which other Cannabinoids are synthesized. It is chemically 2-[(2E)-3,7-dimethyl-2,6-octadien-1-yl]-5-pentyl-1,3-benzenediol.

Cannabigerolic acid (CBGA) is the acidic form of cannabigerol. It is a dihydroxybenzoic acid that is olivetolic acid in which the hydrogen at position 3 is substituted by a geranyl group. A biosynthetic precursor to Delta-tetrahydrocannabinol, the principal psychoactive constituent of the Cannabis plant. It is chemically 3-[(2E)-3, 7-dimethylocta-2, 6-dienyl]-2, 4-dihydroxy-6-pentylbenzoic acid.

Cannabidiol is a phytocannabinoid derived from Cannabis species, which is devoid of psychoactive activity, with analgesic, anti-inflammatory, antineoplastic and chemopreventive activities. It is chemically 2-[(1R,6R)-3-methyl-6-prop-1-en-2-ylcyclohex-2-en-1-yl]-5-pentylbenzene-1, 3-diol.

The Cannabinoids provide various therapeutic benefits like treatment and prevention of Alzheimer's Dementia, Hyperlipidemia, Depression, Anxiety, Inflammatory Disease and Metabolic Disorders.

Before the last century, when it became illegal in most Countries, Cannabis was widely used in medicine. The reason for the restriction in its availability as a therapeutic agent was its growing notoriety as a psychotropic agent and its consequent abuse. In recent years, however, there has been a resurgence in support for the legalization of Cannabinoids for medical use as a result of media attention as well as expectations of their efficacy, albeit, this is not always supported by scientific evidence.

There are currently several methods of cannabinoid delivery for therapeutic benefits. Lung delivery is most commonly achieved by smoking Cannabis. However, there are health concerns for this mode of administration. Cannabis smoke carries even more tars and other particulate matter than tobacco, so it may cause a loss of lung function or cancer. Furthermore, many patients find the act of smoking unappealing, as well as being generally unhealthy. For these reasons, smoking Cannabis is not acceptable as a medical means of administration.

Oral Cannabinoids delivery is useful as it provides easy route of administration. Various formulations are currently available commercially in USA. Dronabinol (Marinol®) is a synthetic tetrahydrocannabinol (THC) which is delivered orally, in sesame oil as capsules. Nabilone (Cesamet®) is a synthetic cannabinoid and an analog of THC and is delivered orally in capsules with povidone and corn starch. Nabiximols (Sativex®) is a natural extract of Cannabinoids containing defined amounts of THC and Cannabidiol (CBD) and is delivered as a liquid, by way of an oromucosal spray.

U.S. patent Ser. No. 11/040,017 discloses cannabidiol (CBD) formulation comprising full spectrum cannabidiol oil (FSO), and cannabidiol isolate (CBD isolate), wherein the FSO and CBD isolate contribute an amount of CBD to the formulation, wherein the amount of CBD is 60-75% (wt.) of the formulation, and wherein the formulation contains less than 7% (wt.) tetrahydrocannabinol (THC).

US patent application number 20210186870 discloses a cannabinoid formulation for oral administration, comprising: one or more Cannabinoids selected from the group consisting of:

0.1-100 mg tetrahydrocannabinol (THC);

0.1-750 mg tetrahydrocannabinolic acid (THCA);

0.1-750 mg cannabidiol (CBD);

0.1-750 mg cannabidiolic acid (CBDA); and

0.1-750 mg cannabigerol (CBG);

0.1-750 mg cannabinchromene (CBC);

and

a lipid carrier comprising or consisting of camelina oil.

U.S. patent Ser. No. 10/307,392 discloses method of treating with reduced adverse psychotropic effect pain, inflammation, mood and motor symptoms associated with psychiatric, autoimmune and neurological diseases and disorders in a subject manifesting one or more of said symptoms,

the method comprising administering to a subject 0.3 mg or more of a pharmaceutical compound one or more times in 24 hours, comprising of

an extract of a Cannabis plant and at least one pharmacologically inactive substance; and

where said extract contains one or more tetrahydrocannabinol (THC)

isomers and one or more cannabidiol (CBD) isomers in the ratio of 1 (THC) and 2 (CBD) by mass; and

where said CBD augments the pharmacodynamics of THC in a subject; and

where said compound contains one or more non-cannabinoid components consisting of one or more of the following: terpenes; sterols; triglycerides; alkanes; squalene; tocopherol; carotenoids; chlorophyll; flavonoid; glycosides and alkaloids.

US publication number 20190255014 discloses topical formulation, comprising: (a) a therapeutically effective amount of at least one cannabinoid compound; (b) at least one skin protecting/enhancing ingredient; and (c) a pharmaceutical carrier effective for simultaneous transdermal delivery of said at least one cannabinoid compound and topical delivery of said skin protecting/enhancing ingredient.

US publication number 20210212983 discloses an oral formulation comprising at least one cannabinoid and at least one mouthfeel experience enhancer.

The uses of Cannabinoids in combination with other herbal ingredients provide better options as far as therapeutic benefits are concerned.

PCT application number WO2020044116 discloses oral formulation comprising: a. one or more cannabinoids selected from among the group consisting of:

0.1-750 mg tetrahydrocannabinolic acid (THCA),

0.1-100 mg tetrahydrocannabinol (THC),

0.1-750 mg cannabidiolic acid (CBDA),

0.1-750 mg cannabidiol (CBD),

0.1-750 mg cannabichromene (CBC),

0.1-750 mg cannabigerol (CBG); and

b. at least one of: and any other oid.

PCT application number WO2015184127 discloses a number of different oral formulations including: an alcohol free formulation in which the cannabinoid is formulated in a mix of polyethylene glycol and propylene glycol, optionally with water, a formulation containing alcohol and a formulation containing lipids. In each of the formulations disclosed, the cannabinoid is a synthetically produced (as opposed to a naturally extracted) cannabidiol.

However, the commercially available cannabinoid delivery systems have erratic absorption and poor bioavailability. This is in part due to factors such as, poor aqueous solubility, limited bioavailability, and cannabinoid instability. Therefore, there is real unmet medical need for improved modes of cannabinoid delivery.

Due to prevalence of drug testing for THC in workplace, consumers wish to take formulations derived from isolates of CBD and CBG. Such formulations are devoid of terpenes, which are important for the ‘entourage effect’. Since Cannabis Terpenes are identical to other plant terpenes, it is possible to combine terpenes from Essential Oils of plants such as Nutmeg and Black pepper, to deliver zero THC formulations with full ‘entourage effect’ to obtain desired therapeutic effect. This methodology of combining specific plant Terpenes with Cannabinoids does not exist to provide better options for consumers.

The present invention provides a polyherbal dispersible composition which is useful for oral delivery of Cannabinoids.

### OBJECTS OF THE INVENTION

An object of the present invention is to provide a polyherbal Pan PPAR and ECS Agonist composition comprising Cannabinoids such as PEA in combination with other botanical extracts suitable for oral administration. Oral administration is in liquid or capsule format.

Yet another objective is to combine PEA with botanical extracts from plants such as Nutmeg, Black Pepper, Holy Basil, Cinnamon, Cassia, Ginger, Rosemary, Terpene isolates of Beta Caryophyllene and Limonene. The total content of botanical extracts and Terpenes is between 2 to 6% by volume of the total formulation in liquid dosage, and up to 30% in solid dosage form, thereby ensuring the entourage effect and improved therapeutic outcomes. Botanical extracts refers to the combination of Essential Oil and Oleoresins.

Yet another object is the combination of Tocotrienols, Carotene Complex, Coenzyme Q10, Vitamin D3, Tocopherols and Vitamin K2-MK7. Tocotrienols are extracted from vegetable oils, including palm oil, rice bran oil, wheat germ, barley, saw palmetto, annatto, and certain other types of seeds, nuts and grains, and the oils derived from them. Carotene complex is sourced either from Oil Palm or Annatto.

Yet another object is the combination with Piperine as a bioavailability enhancer.

Yet another object is the combination with Phyto Cannabinoids derived from Cannabis. Combination with Cannabis is optional, for patients that seek enhanced activation of CB1 and CB2 receptors of Endocannabinoid System in addition to PPAR activation.

Yet another object of the present invention is to provide a composition in combination with Omega-3 fatty acids such as DHA, EPA and/or ALA. Combination with DHA and EPA is optional.

In a further object of the invention, the present invention relates to a method of treating a disease in a subject in need thereof, said method comprising administering the subject a polyherbal water dispersible SMEDS or Liposomal composition comprising Cannabinoids in combination with other herbs.

Other objects and benefits of the present invention will be more apparent from the following description, which is not intended to bind the scope of the present invention.

### SUMMARY OF THE INVENTION

Accordingly, in an embodiment, there is provided a composition comprising polyherbal water dispersible composition comprising Cannabinoids such as PEA in combination with botanical extracts from plants including Nutmeg, Black Pepper, Holy Basil, Cinnamon, Cassia, Ginger, Rosemary, Terpene isolates of Beta Caryophyllene, Limonene, Tocotrienols, Carotene Complex, Coenzyme Q10, Vitamin D3, Tocopherols, Vitamin K2-MK7 and Piperine.

In an aspect of the embodiment, the composition is Self-microemulsifying drug delivery system (SMEDDS).

In another aspect of the embodiment, the composition is liposomal.

The present invention provides synergistic effects of the ingredients for the management of various diseases.

In an aspect of the embodiment, the composition is administered orally.

In another aspect of the embodiment, the composition is in the form of a solid dosage form. In a specific aspect of the embodiment, the composition is in the form of granules or powder or capsule.

In another aspect of the embodiment, the composition is in the form of a liquid dosage form. In a further aspect of the embodiment, the composition is in the form of solution or suspension.

In a further aspect of the embodiment, the present invention relates to a method of treating a disease in a subject in need thereof, said method comprising orally administering the subject a polyherbal dispersible composition comprising Cannabinoids in combination with other herbs.

### DETAILED DESCRIPTION OF THE INVENTION

The invention will now be described in detail in connection with certain preferred and optional embodiments, so that various aspects thereof may be more fully understood and appreciated.

The terms used in the specification are defined as follows.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

A “subject,” “individual,” or “patient,” is used interchangeably herein, which refers to a vertebrate, preferably a mammal, more preferably a human. Tissues, cells and their progeny of a biological entity obtained in vitro or cultured in vitro are also encompassed.

The use of the terms “a” and “an” and “the” and similar referents in the context of describing the elements (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential to the practice of the invention.

As used herein, the terms “treat,” “treated,” or “treating” mean both therapeutic treatment or prophylactic or preventative measures wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent. For example, a mammal or animal may be in need of treatment or prevention of pain without sedation or without significant sedation.

Docosahexaenoic acid (DHA) is essential for maintenance of normal brain function in adults. The inclusion of plentiful DHA in diet improves learning ability, whereas deficiencies of DHA are associated with deficits in learning. DHA is taken up by the brain in preference to other fatty acids. Decrease in DHA in the brain is usually associated with cognitive decline during aging and with onset of sporadic Alzheimer disease. Epidemiological studies have shown a strong correlation between fish consumption and reduction in sudden death from myocardial infarction. The reduction is approximately 50% with 200 mg per day of DHA from fish. DHA not only reduces triglycerides in the blood and decrease thrombosis, but it also prevents cardiac arrhythmias. The association of DHA deficiency with depression is the reason for the robust positive correlation between depression and myocardial infarction.

Alpha-Linolenic Acid (ALA),is an omega-3 essential fatty acid. ALA is found in many seeds and oils, including chia, flaxseeds many common vegetable oils. ALA can only be obtained by humans through their diets. While Eicosapentaenoic acid (EPA; 20:5, n-3) and Docosahexaenoic Acid (DHA; 22:6, n-3) are readily available from fish and algae oil and play a vital role in many metabolic processes, these can also be synthesized by humans from dietary α-linolenic acid: ALA→stearidonic acid→eicosatetraeonic acid→eicosapentaenoic acid→docosapentaenoic acid→9,12,15,18,21-tetracosapentaenoic acid→6,9,12,15,18,21-yetracosahexaenoic acid→docosahexaenoic acid, but with an efficiency of only a few percent. Because the efficacy of n-3 long-chain polyunsaturated fatty acid (LC-PUFA) synthesis decreases down the cascade of ALA conversion, DHA synthesis from α-linolenic acid is even more restricted than that of EPA. Conversion of ALA to DHA is higher in women than in men. The present invention includes an ALA formulation alternative to those that do not wish to take DHA. The invention aims to improve the conversion of ALA to DHA and EPA through a water dispersible formulation and presence of other ingredients through which a synergistic mechanism of action are realized.

Palmitoylethanolamide (PEA) has emerged as a potential nutraceutical, because this compound is naturally produced in many plant and animal food sources, as well as in cells and tissues of mammals, and endowed with important neuroprotective, anti-inflammatory and analgesic actions. Several studies demonstrated that PEA can act via direct activation of at least two different receptors: the PPAR-α and the orphan GPCR 55 (GPR55). As a result, the theory of the ‘entourage’ effect was put forward to raise the possibility that PEA could produce indirect receptor-mediated effects. For example, PEA, through the inhibition of the expression of FAAH, the enzyme responsible for the degradation of the endogenous cannabinoid receptor ligand (or endocannabinoid), anandamide (AEA), may indirectly activate CB2 and CB1 receptors. Likewise, PEA can indirectly activate the transient receptor potential vanilloid receptor type 1 (TRPV1) channels, which are also targets for the endoCannabinoids. In addition, PEA is also able to increase AEA- or 2-AG-induced TRPV1 activation and desensitization. More recently, it has also been demonstrated that PEA can activate TRPV1 channels or increase the expression of CB2 receptors via PPAR-α receptors. In summary, these results suggest that PEA does not operate through just one main mechanism of action. Instead, synergistic interactions among several mechanisms often seem necessary so that PEA can produce its important therapeutic effects, both in the central and the peripheral nervous system.

The anti-inflammatory effects of PEA seem to be mainly related to its ability to modulate mast cell activation and degranulation, and this action is also known as the ALIA (autacoid local inflammation antagonism) mechanism. it was later shown that PPAR-α also mediates the anti-inflammatory effects of PEA, although a direct activation of GPR55 or PPAR-α occurs, PEA can produce its anti-inflammatory action in the gut also via indirect activation of CB1 and CB2 receptors, probably due to the ability of this compound to potentiate the action of endoCannabinoids at these receptors. Both the pharmacological studies as well as the clinical trials supported PEA's action as an analgesic compound.

Leading researchers at U.S. National Institutes of Health (NIH) recognize the importance of the endocannabinoid system as a crucial fulcrum of health. This is emphasized in a 2013 report by NIH scientists Pal Pacher and George Kunos, who maintain that “modulating endocannabinoid system activity may have therapeutic potential in almost all diseases affecting humans”. Endocannabinoid System plays a critical role in regulation of disease. Modulating Endocannabinoid System activity may have therapeutic potential in almost all diseases affecting humans, including obesity/metabolic syndrome, diabetes and diabetic complication, neurodegenerative, inflammatory, cardiovascular, liver, gastrointestinal, skin diseases, pain, psychiatric disorders, cachexia, cancer, chemotherapy induced nausea and vomiting, among many others.

CBD works indirectly, stimulating the body's endogenous cannabinoid system by blocking or inhibiting the FAAH enzyme responsible for breaking down anandamide. When more anandamide is present, there is greater CB1 activation and a more vital endocannabinoid system. CBD acts through multiple receptor-independent channels and it also binds to various receptors in the brain, including serotonin 5HT1A (which contributes to CBD's anti-anxiety effect), TRPV1 (which contributes to CBD's anti-psychotic effect), the nuclear receptor PPAR-gamma (regulates gene expression), and the orphan receptor GPR55 (contributing to its anti-cancer and osteoprotective effects), among others. Cannabinoids such as THCA, CBDA, CBG and CBGA are potent COX Enzyme inhibitors.

CBD minimizes the psychotropic effects of THC without lowering blood or tissue levels of THC. The reversal by CBD of some of the undesirable effects produced by pure THC strengthens the view that medicinal cannabis containing reasonably high levels of CBD is a better drug than cannabis with low levels of CBD or pure THC alone.

Cannabidiol Promotes Browning in 3T3-L1 Adipocytes. Cannabidiol Attenuates Cardiac Dysfunction, Oxidative Stress, Fibrosis, Inflammatory and Cell Death Signaling Pathways in Diabetic Cardiomyopathy.

Use of unheated cannabis extract rich in acidic phytocannabinoids such as CBDA may beneficially affect the uptake and metabolism of CBD or other phytocannabinoids.

The neuroprotective potential of CBD, based on the combination of its anti-inflammatory and anti-oxidant properties, acts not only through the endocannabinoid system, but also causes direct or indirect activation of metabotropic receptors for serotonin or adenosine, and can target nuclear receptors of the PPAR family and also ion channels.

The beta amyloid (Aβ) and other aggregating proteins in the brain increase with age and are frequently found within neurons. The mechanistic relationship between intracellular amyloid, aging and neurodegeneration is not, however, well understood Aβ induces the expression of multiple proinflammatory genes and an increase in both arachidonic acid and eicosanoids, including prostaglandins that are neuroprotective and leukotrienes that potentiate death.

The word “cannabis” refers to a genus of flowering plants. Plants of genus cannabis include several species, including Cannabis sativa, Cannabis indica, and Cannabis ruderalis. There is a long history of cultivating plants of genus cannabis for hemp fibers, seeds and seed oils, medicinal purposes, and recreational activities.

Cannabinoids such as tetrahydrocannabinol stimulate the removal of intraneuronal Aβ, block the inflammatory response, and are protective. Altogether these data show that there is a complex and likely autocatalytic inflammatory response within nerve cells caused by the accumulation of intracellular Aβ, and that this early form of proteotoxicity can be blocked by the activation of cannabinoid receptors.

Cannabinoids receive increasing interest as analgesic treatments. Cannabinoids such as Cannabidiol (CBD) and Δ(9)-tetrahydrocannabinol (THC) & CBG interact with transient receptor potential (TRP) channels and enzymes of the endocannabinoid system. These results are relevant to the analgesic, anti-inflammatory and anti-cancer effects of Cannabinoids and Cannabis extracts.

Vitamin D is a group of fat-soluble secosteroids responsible for increasing intestinal absorption of calcium, magnesium, and phosphate, and many other biological effects. In humans, the most important compounds in this group are vitamin D3 (also known as cholecalciferol) and vitamin D2 (ergocalciferol).

Nuclear receptor family is divided into two subfamilies. The first group includes the estrogen, androgen, progesterone and mineralocorticoid receptors, and the second group includes vitamin D receptor (VDR), the thyroid receptor (TR), retinoic acid receptor (RAR), retinoid X receptor (RXR), and peroxisome proliferator-activated receptors (PPARs). The second group of receptors can form heterodimers with each other, and can function through interacting with appropriate ligands at genetic level. In particular, the PPARs and/or like the VDR represent a major research target for the understanding and treatment of many diseases.

It has been demonstrated that PPARs, mainly the isoforms a and 7, concur in mediating the metabolic and anti-inflammatory effects of (endo) cannabinoid molecules, towards which they exhibit a different selectivity profile. In this regard, it has been demonstrated that synthetic and plant-derived Cannabinoids attenuate neuroinflammation and neurodegeneration in animal models of acute or chronic neurodegenerative disorders through activation of cannabinoid receptors and PPARγ pathway. While natural and synthetic phytocannabinoids including Δ9-tetrahydrocannabinol (Δ9-THC), Cannabidiol (CBD), Δ9-THC acid, ajulemic acid, quinone derivatives, and the recently identified cannabimovone are PPARγ agonists, the acidic derivatives cannabigerolic and cannabidiolic acids exhibit a dual agonist profile. The endocannabinoid anandamide (AEA) activates both PPARα and PPARγ, albeit its efficacy and potency toward PPARα are higher in comparison to PPARγ. The endocannabinoid-like compounds OEA and PEA, structurally related to AEA, do not bind endocannabinoid receptors with high affinity but exert their metabolic and anti-inflammatory effects mainly through PPARα activation.

The formulation outlined in this disclosure has a Water Dispersible mean particle size of 70.22 Micrometers as calculated using volume weighted mean method and 1.11 Micrometer as calculated using number weighted mean method. Particle size was measured using Single Particle Optical Sensing (SPOS) technique. In an aspect, the formulation is Water Dispersible formulation.

The accurate selection of Water Dispersible preparation method depends the physicochemical characteristics of the material to be entrapped, choice of Water Dispersion ingredients, nature of the medium in which the lipids are to be dispersed, effective concentration of the entrapped substance, optimum size and shelf life of the vesicle, and batch-to-batch reproducibility.

Accordingly, in an embodiment, there is provided a composition comprising polyherbal water dispersible composition comprising Cannabinoids such as PEA in combination with botanical extracts from plants including Nutmeg, Black Pepper, Holy Basil, Cinnamon, Cassia, Ginger, Rosemary, Terpene isolates of Beta Caryophyllene, Limonene, Tocotrienols, Carotene Complex, Coenzyme Q10, Vitamin D3, Tocopherols, Vitamin K2-1VK7 and Piperine.

TABLE 1
Composition according to the invention
Composition
Serving Size: Half Tablespoon (7.5 mL) for Oral or Solid/Capsule

| Active Ingredients | Amount per Serving | | PPAR and ECS Activation |
|---|---|---|---|
| PEA | 300 mg-1000 mg | | PPAR α (alpha) and ECS |
| COQ10 | 100 | mg | PPAR α (alpha) and PPAR γ (gamma) |
| Tocotrienols | 50 mg-100 mg | | PPAR α, PPAR γ and PPAR δ (Delta) |
| Vitamin A (Carotene) | 900 | mcg RAE | PPAR γ (gamma) |
| Vitamin K2-MK7 | 150 | mcg | PPAR α (alpha) |
| Vitamin D3 | 2000 | IU | Vitamin D Receptor crosstalk with PPAR |
| Tocopherols | 12-15 | mg | PPAR γ (gamma) |
| Piperine | 5 mg-10 mg | | PPAR γ (gamma) |
| Alpha Linoleic Acid | 2000 | mg | PPAR α (alpha) and PPAR γ (gamma) |

Botanical Extract and Terpenes:

| | | | |
|---|---|---|---|
| Rosemary Extract | 100 | mg | PPAR γ (gamma) |
| Ginger Extract | 50-100 | mg | PPAR γ (gamma) |
| Cassia/Cinnamon Extract | 50-100 | mg | PPAR γ and PPAR δ (Delta) |
| Holy Basil Extract | 50-100 | mg | PPAR γ (gamma) |
| Nutmeg Essential Oil | 25-50 | mg | PPAR γ (gamma) |
| Black Pepper Extract | 25-50 | mg | PPAR γ (gamma) |
| Beta Caryophyllene | 15-25 | mg | ECS (CB2) and PPAR γ (gamma) |
| Limonene | 10-20 | mg | PPAR α (alpha) |

Optional Ingredients:

| CBD | 0 mg-200 mg | | PPAR γ (gamma) and ECS |
|---|---|---|---|
| THC | 0-10 | mg | PPAR γ (gamma) and ECS |
| CBG | 0 mg-200 mg | | PPAR γ (gamma) and ECS |
| CBN | 0-10 | mg | PPAR γ (gamma) and ECS |
| DHA (replaces 1000 mg ALA) | 1000 | mg | PPAR α (alpha) and PPAR γ (gamma) |

To allow titration or dose adjustment of DHA, DHA is replaced with Alpha Linolenic Acid (ALA) derived from Chia and/or Flax and Perilla Seeds according to the invention. In Solid Dose format, ALA is completely replaced with DHA, in such instances the formulation is prepared with only 1000 mg of DHA and without ALA.

The Water Dispersible composition according to the invention contains emulsifiers.

The commercially available emulsifiers containing Sodium Stearoyl Lactylate (E481) and CITREM (E472c) is used to achieve water dispersibility. Alternatively, Polyglycerol polyricinoleate (E476), Lecithin (sunflower or Soy E322), Polyglyceryl-10 Mono/Dioleate or Polysorbates (Tween 80, 20 ect) can also be used.

The mixture is homogenized using a high shear homogenizer, ultrasonic homogenizer or high pressure homogenizer.

In an aspect of the embodiment, the composition is administered orally.

In another aspect of the embodiment, the composition is in the form of a liquid dosage form. In a further aspect of the embodiment, the composition is in the form of solution or suspension.

The compositions according to the present invention provides effects like Pan PPAR and ECS Agonist composition with Antioxidant and Anti-inflammatory function to treat and prevent Chronic Pain, Alzheimer's Dementia, Hyperlipidemia, Depression, Anxiety, Inflammatory Disease and Metabolic Disorders.

The compositions comprise one or more inert excipients. The term “inert excipients”, denotes any of the components of a composition other than the active and which are approved by regulatory authorities or are generally ‘regarded as safe’ for human or animal use. A combination of excipients may also be used. The amount of excipient(s) employed will depend upon how much active agent is to be used. One excipient can perform more than one function.

The pharmaceutical compositions of the invention can be prepared combining an active pharmaceutical ingredient of the invention with an appropriate pharmaceutically acceptable carrier, diluent or excipient, and may be formulated into preparations in solid or liquid forms, such as tablets, capsules, powders, granules and microspheres.

In another embodiment, the present invention relates to administering “an effective amount of the composition of invention” to the subject suffering from said disease. Accordingly, active pharmaceutical ingredient and pharmaceutical compositions containing them may be administered using any amount, any form of pharmaceutical composition via any route of administration effective for treating the disease. Typical routes of administering such pharmaceutical composition includes oral route.

Pharmaceutical compositions of the invention are formulated so as to allow the active ingredients contained therein to be bioavailable upon administration of the composition to a patient. Compositions that will be administered to a subject or patient may take the form of one or more dosage units. The dosage forms can also be prepared as sustained, controlled, modified and immediate dosage forms.

Pharmaceutical compositions of the invention are formulated so as to allow the active ingredients contained therein to be combined with other Pharmaceutical ingredients such as Statins, Kratom Alkaloids and Psilocybin extracts.

The foregoing examples are illustrative embodiments and are merely exemplary. A person skilled in the art may make variations and modifications without deviating from the spirit and scope of the invention. All such modifications and variations are intended to be included within the scope of the claims.

Manufacturing Process

Production batches are 15 liters or roughly 63 bottles. Per bottle volume is 237 mL (8 FL Oz). All ingredients in the proportion shown in the following tables are mixed using either a high shear homogenizer, ultrasonic homogenizer or high pressure homogenizer.

Botanical Extracts are a mixture of Essential Oils and Oleoresins are first premixed as follows:

TABLE 2
Botanical Extract Composition according to the invention

| | | Batch Size: | Bottles |
|---|---|---|---|
| Botanical Extract Composition | Dose/ | Per | Per |
| Ingredient | Serving | Bottle | Batch |
| Rosemary Oleoresin | 50 mg | 1.6 g | 100.8 g |
| Ginger Oleoresin | 50 mg | 1.6 g | 100.8 g |
| Cinnamon and Cassia | 50 mg | 1.6 g | 100.8 g |
| Essential Oil | | | |
| Holy Basil | 50 mg | 1.6 g | 100.8 g |
| Essential Oil | | | |
| Rosemary | 25 mg | 0.8 g | 50.4 g |
| Essential Oil | | | |
| Nutmeg | 25 mg | 0.8 g | 50.4 g |
| Essential Oil | | | |
| Black Pepper EO | 25 mg | 0.8 g | 50.4 g |
| Total: | 275 mg | 8.8 g | 554.4 g |
| Total Essential Oil: | 175 mg | 5.6 g | |

The table 2 shows a total of 554.4 grams of Botanical Extracts premixed for a production batch volume of 63 Bottles.

Tocotrienol concentration was roughly 40% and Carotene concentration was 16500 mcg per gram. K2-MK7 concentration was 1000 mcg/gram. Vitamin D3 concentration was 266667 IU/mL. THC Crude had a THC concentration of 9.59% and CBD concentration of 50.12 CBD Isolate had a CBD concentration of 99.8%. MCT oil was used as a filler/excipient. All ingredients in proportions shown in the tables bellow are homogenized along with botanical extracts. Typical overages used was up to 10%. Sodium Stearoyl Lactylate (E481) or SSL is used to achieve water dispersibility, amounting to a maximum of 1% w/w per bottle (2.37 grams maximum limit), are first diluted in warmed MCT oil at 60 Deg C before being added.

### Example 1: Cannabis Free Formulation According to the Invention

TABLE 3
Cannabis Free Formulation according to the invention

| Non-Cannabis | CBD | THC | | | |
|---|---|---|---|---|---|
| 600 mg PEA/Serv | 0 | 0 | | | |
| | Batch Size | 63 | Bottles | 237 | ml/Bot |
| | Lecithin | 12.50% | | | |
| | Emulsifier | 2.00% | | | |
| | Vit D3 Dilution | 266667 | IU/ml. | | |
| | Flax Oil Ratio | 0.8 | | | |
| Ingredient | Per Bottle | Unit | Per Batch | Unit | State |
| CBD (Isolate) | 0.00 | g | 0.0 | g | S |
| THC (Crude) | 0.00 | g | 0.0 | g | S |
| PEA | 19.20 | g | 1209.6 | g | S |
| COQ10 | 3.20 | g | 201.6 | g | S |
| Tocotrienols | 3.52 | g | 221.8 | g | S |
| Carotene | 1.74 | g | 109.9 | g | S |
| K2-MK7 | 4.80 | g | 302.4 | g | S |
| Piperine | 0.32 | g | 20.2 | g | S |
| D3 | 64000.00 | IU | 15.1 | mL | L |
| Botanical Extract | 8.80 | g | 554.4 | g | L |
| Beta Caryophyllene | 0.48 | g | 30.2 | g | |
| Limonene | 0.32 | g | 20.2 | g | |
| MCT Oil | 48.00 | mL | 3024.0 | mL | L |
| Flax Oil | 192.00 | mL | 12096.0 | mL | |
| Lecithin | 30.00 | g | 1890.0 | g | L |
| Emulsifier (SSL) | 2 | g | 126 | g | L |

The above table shows ingredients used to manufacture a Cannabis free version, with 63 Bottles per Batch. CBD and THC content shows zero. Botanical Extracts premixed is used as shown.

### Example 2: THC Free Formulation with CBD Isolate According to the Invention

TABLE 4
THC Free Formulation with CBD Isolate according to the invention

| | CBD | THC | (2000:0) | 9.59% | 50.12% |
|---|---|---|---|---|---|
| | 2000 | 0 | Crude Composition | THC | CBD |
| | Batch Size | 63 | Bottles | 237 | mL/Bot |
| | Lecithin | 12.50% | | | |
| | Emulsifier | 2.00% | | | |
| | Vit D3 Dilution | 266667 | IU/mL | | |
| | Flax Oil Ratio | 0.8 | | | |
| Ingredient | Per Bottle | Unit | Per Batch | Unit | State |
| CBD (Isolate) | 2.00 | g | 126.0 | g | S |
| THC (Crude) | 0.00 | g | 0.0 | g | S |
| PEA | 9.60 | g | 604.8 | g | S |
| COQ10 | 3.20 | g | 201.6 | g | S |
| Tocotrienols | 3.52 | g | 221.8 | g | S |
| Carotene | 1.74 | g | 109.9 | g | S |
| K2-MK7 | 4.80 | g | 302.4 | g | S |
| Piperine | 0.32 | g | 20.2 | g | S |
| D3 | 64000.00 | IU | 15.1 | mL | L |
| Botanical Extracts | 8.80 | g | 554.4 | g | L |
| Beta Caryophyllene | 0.48 | g | 30.2 | g | |
| Limonene | 0.32 | g | 20.2 | g | |
| MCT Oil | 48.00 | mL | 3024.0 | mL | L |
| Flax Oil | 192.00 | mL | 12096.0 | mL | |
| Lecithin | 30.00 | g | 1890.0 | g | L |
| Emulsifier (SSL) | 2.00 | g | 126.0 | g | L |

The above table shows ingredients used to manufacture a THC free version, with 63 Bottles per Batch. THC crude is not used, content shows zero. Botanical Extracts premix is used as shown.

### Example 3: Full Spectrum Formulation with 3000 mg CBD: 300 mg THC According to the Invention

TABLE 5
Full Spectrum Formulation with 3000 mg CBD:
300 mg THC according to the invention

| | CBD | THC | (3000:300) | 9.59% | 50.12% |
|---|---|---|---|---|---|
| | 3000 | 300 | Crude Composition | THC | CBD |
| | Batch Size | 63 | Bottles | 237 | mL/Bot |
| | Lecithin | 12.50% | | | |
| | Emulsifier | 2.00% | | | |
| | Vit D3 Dilution | 266667 | IU/ml | | |
| | Flax Oil Ratio | 0.8 | | | |
| Ingredient | Per Bottle | Unit | Per Batch | Unit | State |
| CBD (Isolate) | 1.43 | g | 90.22 | g | S |
| THC (Crude) | 3.13 | g | 197.08 | g | S |
| PEA | 9.60 | g | 604.8 | g | S |
| COQ10 | 3.20 | g | 201.6 | g | S |
| Tocotrienols | 3.52 | g | 221.76 | g | S |
| Carotene | 1.74 | g | 109.87 | g | S |
| K2-MK7 | 4.80 | g | 302.4 | g | S |
| Piperine | 0.32 | g | 20.16 | g | S |
| D3 | 64000 | IU | 15.12 | mL | L |
| Botanical Extracts | 8.8 | g | 554.4 | g | L |
| Beta Caryophyllene | 0.5 | g | 30 | g | |
| Limonene | 0.3 | g | 20 | g | |
| MCT Oil | 48 | mL | 3024 | mL | L |
| Flax Oil | 192 | mL | 12096 | mL | |
| Lecithin | 30 | g | 1890 | g | L |
| Emulsifier (SSL) | 2 | g | 126 | g | L |

The above table shows ingredients used to manufacture a Full Spectrum THC version, with 63 Bottles per Batch. THC crude is used, content shows quantity used. Botanical Extracts premix is used as shown.

### Example 4: Full Spectrum Formulation with 3000 mg CBD: 450 mg THC According to the Invention

TABLE 6
Full Spectrum Formulation with 3000 mg CBD:
450 mg THC according to the invention

| | CBD | THC | (3000:450) | 9.59% | 50.12% |
|---|---|---|---|---|---|
| | 3000 | 450 | Crude Composition | THC | CBD |
| | Batch Size | 63 | Bottles | 237 | mL/Bot |
| | Lecithin | 12.50% | | | |
| | Emulsifier | 2.00% | | | |
| | Vit D3 Dilution | 266667 | IU/ml | | |
| | Flax Oil Ratio | 0.8 | | | |
| Ingredient | Per Bottle | Unit | Per Batch | Unit | State |
| CBD (Isolate) | 0.65 | g | 40.8 | g | S |
| THC (Crude) | 4.69 | g | 295.6 | g | S |
| PEA | 9.60 | g | 604.8 | g | S |
| COQ10 | 3.20 | g | 201.6 | g | S |
| Tocotrienols | 3.52 | g | 221.8 | g | S |
| Carotene | 1.74 | g | 109.9 | g | S |
| K2-MK7 | 4.80 | g | 302.4 | g | S |
| Piperine | 0.32 | g | 20.2 | g | S |
| D3 | 64000 | IU | 15.1 | mL | L |
| Essential Oils | 8.8 | g | 554.4 | g | L |
| Beta Caryophyllene | 0.48 | g | 30 | g | |
| Limonene | 0.32 | g | 20 | g | |
| MCT Oil | 48.0 | mL | 3024 | mL | L |
| Flax Oil | 192.0 | mL | 12096 | mL | |
| Lecithin | 30 | g | 1890 | g | L |
| Emulsifier (SSL) | 2 | g | 126 | g | L |

The above table shows ingredients used to manufacture a Full Spectrum THC version, with 63 Bottles per Batch. THC crude is used, content shows quantity used. Botanical Extracts premix is used as shown.

### Example 5: Clinical Evaluation of Botanical Pan PPAR Agonist with Cannabinoids as Monotherapy and Adjuvant Therapy with Opioids for Treating Pain, Inflammation and Anxiety

Open label study was conducted by Dr Eric Ehlenberger MD, Accurate Clinic, 2401 Veterans Memorial Blvd #16, Kenner, LA 70062. Correspondence to Dr. Eric Ehlenberger:doctor@ehlenberger.com. The study subjects, patients in a chronic pain management program, were offered a supplement presented as possibly offering benefits for chronic pain, anxiety and sleep. The subjects electing to participate in the study included those who suffer from inflammatory diseases including osteoarthritis, rheumatoid arthritis and interstitial cystitis as well as those with chronic headaches, neck and low back pain, sciatica, peripheral neuropathy and fibromyalgia. Forty-one patients elected to participate in the trial. All patients in the study group were currently taking prescription opioids at dosages ranging from 30 to 285 morphine equivalents (ME) per day. The opioids were either taken as stand-alone medications or taken with adjuvant medications including gabapentinoids, antidepressants, NSAIDs and/or medical marijuana products including CBD and THC-based formulations. During the study period one patient obtained a lumbar epidural steroid injection for low back pain associated with severe sciatica. The subjects were queried on their follow-up pain management visit one month after initiating use of their pan-PPAR supplement as to any benefits identified with use of the supplement. Patients self-reported perceived clinical benefits including reduction of pain and/or anxiety and improvement of insomnia. Additionally, when applicable, patients reported whether use of the formulation allowed for sparing of the use of therapeutic opioids or medical cannabis. Additionally, subjects were asked how use of their experimental formulation compared to previous experience of benefits associated with the use of other CBD-based OTC products.

The trial formulations contain multiple botanical constituents which provide anti-inflammatory and antioxidant effects via different mechanisms of action. While many of the included constituents have complex, multi-mechanistic therapeutic activities, a common denominator of many of the constituents which contributes to the novelty of these formulations is PPAR activation. The synergy of their activation of different PPAR isoforms is proposed to contribute significantly to the markedly beneficial therapeutic responses to these formulations. PPAR γ isoforms are activated by a blend of proprietary botanical terpenes and hemp-derived cannabinoids including CBD and THC while PPAR a isoforms are activated by palmitoylethanolamide (PEA). Additional activation of PPAR β/δ isoforms is provided by cinnamaldehyde.

Antioxidant activity is provided by beta carotene, CoQ10 and the tocotrienols. Antiinflammatory activity is provided by omega-3, PEA and cannabinoids. Bio-availability enhancement is achieved with water dispersible Self Micro Emulsifying Delivery System (SMEDDS). Having a family of formulations with multiple constituents to achieve synergistic agonism of multiple receptors including PPARS, CB1, CB2, 5HT, TRPV1 and GPR offers improved efficacy as all these receptors impact both pain and inflammation.

Based on their pan-PPAR agonism mechanisms of action, formulations that are inclusive of CBD, THC, PEA and other botanical terpenes offer potentially significant synergistic benefits for analgesia, anxiety and insomnia. Additional benefits appear to include the potential to reduce reliance on opioids and cannabis use. When combined with botanical antioxidants such as omega-3, beta carotene, tocotrienols and CoQ10, there may be additional long term benefits for reducing chronic pain related to neuroinflammation and central sensitization as well as a reduction or limited progression of opioid analgesic tolerance.

The four formulations from Carolina Cannabinoids:

1. Anti-Inflammatory & Antioxidant Complex with Broad Spectrum 2000 mg CBD (THC free) per 240 ml bottle [Example 2 according to the invention]
2. Anti-Inflammatory & Antioxidant Complex with Full Spectrum (3000 mg CBD: 300 mg THC) per 240 ml bottle [Example 3 according to the invention]
3. Anti-Inflammatory & Antioxidant Complex with Full Spectrum (3000 mg CBD: 450 mg THC) per 240 ml bottle [Example 4 according to the invention]
4. Anti-Inflammatory & Antioxidant Complex (no CBD or THC) per 240 ml bottle

### Example 1 According to the Invention

Patients were advised to slowly titrate up the dosing of their formulations, initially starting with a ½ tsp (2.5 ml) oral dose at night then to slowly increase this dose to twice a day then three times a day as tolerated to achieve a target dose of 7.5 mL per day. It was recommended patients take their formulations orally before meals, and swallow with warm water.

### Results and Discussion

Patients reported potent analgesic benefits in those suffering from inflammatory diseases including osteoarthritis, rheumatoid arthritis and interstitial cystitis. Patients with fibromyalgia also reported benefits for their pain, fatigue and non-restful sleep. Many patients throughout the pain spectrum reported improvement with anxiety and insomnia. Opioid sparing benefits were frequently reported, surprisingly including those patients trialing the formulation that included CBD without THC. These opioid sparing benefits were observed with the use of the pan-PPAR agonists in patients taking as little as 30 ME/day or as much as 270ME/day. There appeared to be a trend toward greater opioid sparing benefits with the use of the pan-PPAR agonist formulations that included THC.

One surprise result was reported by a patient taking both opioids and medical marijuana who received a lumbar epidural steroid injection for severe sciatica while taking the pan-PPAR formulation with THC. This patient volunteered an “unexpectedly high degree of pain relief” that surprised both the patient and the interventional pain physician who performed the injection. Given that this patient was already using THC at substantial doses, the benefit he received was postulated as coming from the synergy from the pan-PPAR agonists and CBD.

Later in the study patients were queried as to the presence of cannabis product sparing with the use of the pan-PPAR formulations and a positive correlation was found predominantly in THC containing formulations. Because this question was not presented until later in the study, only 9 patients were queried but of these 9, eight patients noted a reduction in their use of THC-based medical cannabis products, including one patient using the CBD non-THC formulation.

The formulations were well tolerated with minimal adverse effects limited to mild drowsiness, nausea and belching that did not require discontinuation of use.

The results are included in table 7 below for individual patients.

TABLE 7
Patient evaluation data as per the studies in example 5.

| | | | | | Enhanced EF > | Diagnosis/ | Dose | Doses/ |
|---|---|---|---|---|---|---|---|---|
| # | M/F | Age | ME | Formulation | other CBDs | Body Part(s) | (ml) | day |
| 1 | M | 54 | | Example 2 | | Knee | 2.5 | 1 |
| 2 | M | 63 | | Example 2 | | LBP | 2.5 | 1 |
| 3 | M | 64 | 0 | Example 3 | | neck pain | 5 | 1-1.5 |
| 4 | F | 64 | 79 | Example 2 | | RA, neck pain, | 5 | 1-2 |
| | | | | | | cervical | | |
| | | | | | | radiculopathy, | | |
| | | | | | | fatigue | | |
| 5 | M | 56 | 55 | Example 2 | | neck pain, LBP, | 10 | 1 |
| | | | | | | anxiety | | |
| 6 | F | 54 | 60 | Example 3 | | muscle spasm | | |
| 7 | M | 72 | 60 | Example 2 | | knee pain arthritis, | | |
| | | | | | | lumbar arthritis | | |
| 8 | F | 57 | 180 | Example 4 | yes | mid, lower back | 2.5 | 1 |
| 9 | F | 45 | 45 | Example 4 | | neck, lower | 2.5-5 | 1 |
| | | | | | | back, FMS | | |
| 10 | M | 78 | 60-80 | Example 2 | yes | neck, back, | 0.5 | 2 |
| | | | | | | myofacial, sleep | | |
| 11 | F | 53 | 45 | Example 4 | | mood stabilizer, | 0.5 | 2 |
| | | | | | | nerve pain hand | | |
| | | | | | | IC pain & frequency | | |
| 12 | F | 54 | | Example 2 | | neck pain | 7.5 | 1 |
| | | | | | | anxiety sleep | | |
| 13 | M | 61 | 30 | Example 2 | | widespread | 5 | 2 |
| | | | | | | joint pain | | |
| 14 | M | 47 | 30 | Example 2 | | LBP | 7.5 | 1 |
| 15 | F | 62 | 30 | Example 4 | | LBP, knee pain, | 2.5 | 2 |
| | | | | | | arm fracture | | |
| 16 | M | 59 | 40 | Example 4 | | B/L shoulder | 5 | 3 |
| 17 | F | 44 | | Example 3 | | neck, back, | 2.5 | 2 |
| | | | | | | myofacial | | |
| 18 | M | 58 | 50 | Example 3 | | neck, back, | 5 | 1 |
| | | | | | | myofacial | | |
| 19 | M | 64 | — | Example 2 | | anxiety | | |
| 20 | F | 59 | 190 | Example 4 | | lower back, CRPS | 5 | 2 |
| 21 | M | 48 | 135 | Example 2 | | back, DPN | 5 | 2 |
| 22 | F | 39 | 40 | Example 3 | | Neck, LBP, Left | 2.5 | 1 |
| | | | | | | Shoulder, Chronic HA | | |
| 23 | M | 54 | 90 | Example 2 | | Knee pain arthritis, | 5 | 2 |
| 24 | F | 58 | 285 | Example 4 | | myofacial pain, | 5 | 3 |
| | | | | | | neck, LBP | | |
| 25 | M | 53 | | Example 4 | | neck, back, | 0.5 | |
| | | | | | | myofacial | | |
| 26 | | 54 | 45 | Example 3 | | lumbar | 5 | 1 |
| 27 | F | 60 | 30 | Example 3 | | FMS, back, neck | 2.5 | 1 |
| 28 | M | 52 | 65 | Example 3 | | back, myofacial | 10 | 12 |
| | | | | | | pain | | |
| 29 | M | 66 | 60 | Example 4 | | Back, Hip | 5 | 2 |
| 30 | F | 52 | 240 | Example 4 | | back | 2.5 | 1 |
| 31 | M | 72 | 60 | Example 2 | | back | 2.5 | 2 |
| 32 | M | 41 | 0 | Example 4 | | anxiety depression | 7.5 | 1 |
| | | | | | | insomnia | | |
| 33 | F | 59 | 77.5 | Example 2 | | Neck, Back, hips | 5 | 2 |
| 34 | M | 65 | | Example 4 | | Neck, LBP, Right | 5 | 1 |
| | | | | | | Ankle, R Shoulder, | | |
| | | | | | | Bilat Knees. | | |
| 35 | M | 58 | 75 | Example 4 | | Neck, LBP, Shoulder, | 5 | 2 |
| | | | | | | radicular pain, | | |
| | | | | | | sleep, anxiety | | |
| 36 | M | 50 | 30 | Example 3 | yes | LBP, fatigue | 5 | 1 |
| 37 | F | 50 | 270 | Example 4 | yes | LBP, fms pain | 2.5 | 3 |
| | | | | | | anxiety sleep | | |
| 38 | F | 54 | 90 | Example 4 | no | back, neck, | 2.5 | 2 |
| | | | | | | myofacial | | |
| 39 | M | 58 | 90 | Example 4 | yes | neck, back, sleep | 2.5 | 1 |
| 40 | F | 38 | 60 | Example 3 | | abdominal pain, | 5 | 1 |
| | | | | | | gastric parisis | | |
| 41 | F | 50 | 270 | Example 4 | yes | sleep anxiety | 5 | 1 |

| | Benefit | Opioid | Onset | Duration | Satisfaction | |
|---|---|---|---|---|---|---|
| # | 0-10 | Sparing | (Hrs) | (Hrs) | 0-10 | Notes |
| 1 | 8 | yes | 0.5 | 24 | 10 | |
| 2 | 6 | yes | 0.25 | 1 | 5 | |
| 3 | 8.5 | no | 0.5 | 5 | 9 | helps with stress but |
| | | | | | | not pain tastes bad |
| 4 | 10 | yes | 2 | 6 | 10 | “ecstatic” helps |
| | | | | | | prolong benefit of oxy |
| | | | | | | 15 mg. Helps with energy |
| | | | | | | and makes MM work |
| | | | | | | better. Helps with |
| | | | | | | all pain |
| 5 | 7 | yes | 0.75 | 5 | 7 | doesn't need daily |
| 6 | | | | | | |
| 7 | | | | | | |
| 8 | 4 | no | 2-3 | 3 | 4 | needs to take with |
| | | | | | | phenergen, helps with |
| | | | | | | sleep and pain tastes |
| | | | | | | revolting |
| 9 | 7 | no | 5 | 8 | 8 | Effective for pain |
| | | | | | | tastes bad |
| 10 | 0 | no | — | — | 0 | did not notice any |
| | | | | | | benefit tastes bad |
| 11 | 8 | yes | 0.5 | 4 | 8 | hopes to taper down/ |
| | | | | | | off opioids with cannabis |
| 12 | 7 | yes | 0.6 | 8 | | supplement with cannabis |
| | | | | | | flower, reduced need |
| | | | | | | for flower by 40% |
| 13 | | yes | 0.25 | 6 | | very effective |
| 14 | 10 | yes | | | | I don't feel it kicking |
| | | | | | | in but I just know I have |
| | | | | | | less pain tastes bad |
| 15 | 8 | yes | 0.25 | 1 | 7 | good for general pain |
| | | | | | | relief, IN for acute |
| | | | | | | fracture pain in Left arm |
| 16 | 7 | no | 2 | 2 | 7 | prefers over MM for cost |
| 17 | ? | | 1 | 4 | 4 | was able to mow grass for |
| | | | | | | first time in a while, |
| | | | | | | helps with energy |
| | | | | | | burping tastes like |
| | | | | | | basil and cinnamon |
| 18 | 0 | no | | na | 0 | |
| 19 | | | | | | somewhat effective |
| | | | | | | for anxiety |
| 20 | 6 | no | ? | ? | | made me feel “foggy” |
| | | | | | | and “sleepy” |
| 21 | | 0 | | | 0 | |
| 22 | 6.5 | No | 0.3 | 5 | 7.5 | Since using has noticed |
| | | | | | | that sleep has improved |
| | | | | | | in amount of sleep, |
| | | | | | | getting quality sleep |
| 23 | 8 | yes | 1 | 12 | 8 | |
| 24 | 8 | no | 0.5 | 2 | 8 | helps somewhat with |
| | | | | | | anxiety, mostly |
| | | | | | | with pain |
| 25 | 5 | no | | | | Patient says he prefers |
| | | | | | | to use THC 900 tincture + |
| | | | | | | OTC CBD. Patient was |
| | | | | | | only taking ½ |
| | | | | | | of a dropper instead |
| | | | | | | of .5-1 tsp. Will retry |
| 26 | 0 | no | — | — | 0 | |
| 27 | 0 | no | — | — | 0 | |
| 28 | 6 | no | | 8 | 6 | puts in veggie capsules |
| 29 | 5 | no | ? | 3 | | helps “relax” but |
| | | | | | | not with pain |
| 30 | ? | no | ? | ? | ? | only used a few times, |
| | | | | | | helps to settle stomach, |
| | | | | | | effective for back pain, |
| | | | | | | IN for neck myofascial |
| | | | | | | pain/tension HA |
| 31 | 8 | no | ? | ? | | did not help for back pain |
| | | | | | | but with curcumin helped |
| | | | | | | for foot pain (NP?) |
| | | | | | | Patient did not notice any |
| | | | | | | benefit until after a week |
| | | | | | | of taking it |
| 32 | 10 | NA | 3 | 24 | | discontinued use of flower |
| | | | | | | cannabis and SSRI |
| | | | | | | antidepressant |
| 33 | 4 | no | 2 | 8 | 4 | takes HS, waking up less |
| | | | | | | often with pain, moderately |
| | | | | | | effective for hip arthritis |
| | | | | | | tastes bad |
| 34 | 8 | yes | 0.5 | 4 | 9 | Said it works great when |
| | | | | | | using with pain medication |
| | | | | | | but taste horrible. works |
| | | | | | | better than all previous |
| | | | | | | OTC CBD products |
| 35 | 8 | yes | 0.5 | 5 | 8 | Recommended use of CBD- |
| | | | | | | 2000 in daytime and use |
| | | | | | | of CBD/THC-450 works |
| | | | | | | better than LA CBD/ |
| | | | | | | THC tincture |
| 36 | 6 | no | 0.5 | 3 | 8 | |
| 37 | 6 | yes | 0.5 | 4 | 7 | |
| 38 | 7 | yes | 0.5 | 4 | 7 | |
| 39 | 10 | no | 1 | 24 | 10 | helps mostly with sleep, and |
| | | | | | | joint pain. Effective for |
| | | | | | | long covid insomnia and |
| | | | | | | joint aches |
| 40 | 5 | yes | | 1 | 5 | takes the sharp pain away. |
| | | | | | | Patient has bowel absorption |
| | | | | | | issues and doesn't think |
| | | | | | | it absorbs well through J-tube |
| 41 | 10 | yes | 0.5 | 12 | 9 | |

The above compositions may further be combined with
Statins including lovastatin
e.g. Example 1+20 mg Lovastatin and Example 2+20 mg Lovastatin.
Kratom Alkaloids
e.g. Example 1+Ethanol extracted Kratom Alkaloids [between 25 mg to 50 mg of Mitragynine];
   Example 2+Ethanol extracted Kratom Alkaloids [between 25 mg to 50 mg of Mitragynine];
   Example 3+Ethanol extracted Kratom Alkaloids [between 25 mg to 50 mg of Mitragynine];
   Example 4+Ethanol extracted Kratom Alkaloids [between 25 mg to 50 mg of Mitragynine];
Psilocybin
e.g. Example 1+Ethanol extract of Psilocybin [between 5 mg to 15 mg];
   Example 2+Ethanol extract of Psilocybin [between 5 mg to 15 mg];
   Example 3+Ethanol extract of Psilocybin [between 5 mg to 15 mg];
   Example 4+Ethanol extract of Psilocybin [between 5 mg to 15 mg];

What is claimed is:

1. A polyherbal Pan PPAR and ECS Agonist water dispersible composition for Multi-Disease Treatment comprising
PEA
essential oils and extracts from plants such as Nutmeg, Black Pepper, Holy Basil, Cinnamon, Cassia, Ginger, Rosemary, Isolates of Beta Caryophyllene and Limonene;
DHA and or ALA
Carotene Complex
Tocotrienols
Coenzyme Q10
Vitamin K2-MK7
Vitamin D3; and
Cannabinoids derived from Cannabis.

2. The polyherbal composition as claimed in claim 1, wherein the composition is administered orally.

3. The polyherbal composition as claimed in claim 1, wherein the composition is in the form of liquid dosage form.

4. The polyherbal composition as claimed in claim 3, wherein the liquid dosage form is solution or suspension.

5. The polyherbal composition as claimed in claim 1, wherein the composition is in the form of solid dosage form.

6. The polyherbal composition as claimed in claim 5, wherein the solid dosage form is powder or granule or capsule or tablet.

7. The polyherbal composition as claimed in claim 1, wherein the composition provides synergistic activity.

8. The polyherbal composition as claimed in claim 1, wherein the composition is further combined with other active ingredients.

9. The polyherbal composition as claimed in claim 8, wherein the further active ingredients are Lovastatin, Mitragynine or Psilocybin.
